(19) United States
(12) Patent Application Publication
(10) Pub. No.: US 2008/0319203 A1
(43) Pub. Date: Dec. 25, 2008

(54) Method for Producing 5-Fluoro-1,3-Dialkyl-1H-Pyrazol-4-Carboxylic Acid Chlorides

(75) Inventors: Norbert Lui, Odenthal (DE); Sergii Pazenok, Solingen (DE)

Correspondence Address: WOMBLE CARLYLE SANDRIDGE & RICE, PLLC, ATTN: PATENT DOCKETING 32ND FLOOR, P.O. BOX 7037, ATLANTA, GA, 30357-0037, US

(73) Assignee: Bayer CropScience AG, Monheim (DE)

(21) Appl. No.: 12/094,962

(22) PCT Filed: Nov. 23, 2006
(86) PCT No.: PCT/EP2006/011228

§ 371 Date: May 23, 2008

(30) Foreign Application Priority Data
Dec. 1, 2005 (DE) 10 2005 057 180.8

(51) Int. Cl. C07D 231/14

(52) U.S. Cl. 548/374.1

(57) ABSTRACT

The present invention relates to a novel process for preparing known 5-fluoro-1,3-dialkyl-1H-pyrazole-4-carbonyl chlorides which can be used as starting materials for active fungicidal ingredients.

The present invention relates to a novel process for preparing known 5-fluoro-1,3-dialkyl-1H-pyrazole-4-carbonyl chlorides, a valuable precursor for the preparation of fungicides, from the corresponding 5-chloro-1,3-dialkyl-1H-pyrazole-4-carbonyl fluorides.

It is already known that 1,3-dimethyl-5-fluoro-4-carboxamides are obtained by reacting the corresponding acid fluoride with the desired aniline derivative (cf. EP-A 0 776 889). According to this description, preference is given to using bicyclic tertiary amines such as diazabicyclooctane (DABCO), diazabicyclononene (DBN) or diazabicycloundecene (DBU) as an acid acceptor. The reaction with DABCO affords only a yield of 80%. Moreover, DABCO is unsuitable for industrial scale reactions, since this reagent is very expensive and cannot be recycled. Moreover, in the case of use of the acid fluorides, it is disadvantageous that the reaction with the desired aniline derivative, as well as the product, forms an equimolar amount of toxic and very corrosive hydrogen fluoride. As a result, the workup after this reaction is very complicated and expensive for safety reasons.

In contrast, the reaction of acid chlorides with the desired aniline derivative proceeds under mild conditions and in very high yields. A disadvantage of this process is the conversion of acid fluorides to the corresponding acid chlorides by a two-stage process:

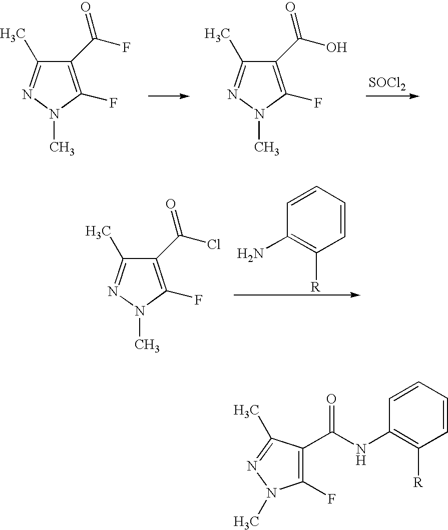

It has now been found that 5-fluoro-1,3-dialkyl-1H-pyrazole-4-carbonyl chlorides of the formula (I)

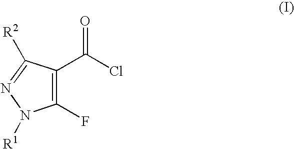

in which R¹ and R² are each independently C₁-C₃-alkyl are obtained by reacting

5-fluoro-1,3-dialkyl-1H-pyrazole-4-carbonyl fluorides of the formula (II)

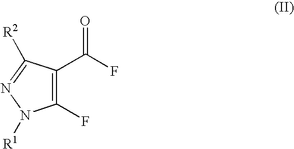

in which R¹ and R² are each as defined above
with a chlorinating agent, optionally in the presence of a diluent.

Surprisingly, the 5-fluoro-1,3-dialkyl-1H-pyrazole-4-carbonyl chlorides of the formula (I) can be prepared with good yields in high purity and selectivity under the inventive conditions. It is a further advantage of the process according to the invention that the reaction can be carried out without any solvents, which has the consequence of a very high space-time yield. The reaction proceeds under very mild conditions (30-50° C.) and enables a fluorine-chlorine exchange in only one step. Since such transformations are unknown for heterocyclic compounds such as pyrazole derivatives, the person skilled in the art would expect that the pyrazole derivative forms a complex, for example with silicon tetrachloride or aluminium trichloride, and not that a fluorine-chlorine exchange takes place.

When, for example, 5-fluoro-1,3-dimethyl-1H-pyrazole-4-carbonyl fluoride and silicon tetrachloride as the chlorinating agent are used, the process according to the invention can be illustrated by the following formula scheme:

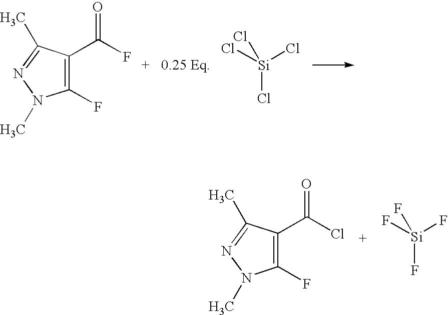

The 5-fluoro-1,3-dialkyl-1H-pyrazole-4-carbonyl fluorides used as starting materials in the performance of the process according to the invention are defined in general terms by the formula (II). The R¹ and R² radicals in this formula (II) are each independently preferably methyl, ethyl, n-propyl or isopropyl, more preferably both methyl.

5-Fluoro-1,3-dialkyl-1H-pyrazole-4-carbonyl fluorides of the formula (II) are known or can be prepared by known processes (cf. EP-A 0 776 889).

In the performance of the process according to the invention, a chlorinating agent is used. Suitable chlorinating agents for this purpose are all chlorinating agents customary for such reactions, especially silicon tetrachloride, trichloromethylsilane, dichlorodimethylsilane, trichlorophenylsilane, aluminium trichloride, boron trichloride, calcium chloride, titanium tetrachloride, tin tetrachloride, zinc dichloride and bismuth trichloride. It is also possible to use mixtures of these chlorinating agents. Mixtures suitable with particular preference are the mixtures of silanes and AlCl₃ or ZnCl₂, for example mixtures of SiCl₄ and AlCl₃, in which case AlCl₃ or ZnCl₂ serves as the catalyst and is used in the amount of from 1 to 3 mol % based on SiCl₄.

The process according to the invention can be carried out in the absence or in the presence of a diluent. Preference is given to working without diluents. However, it is also possible to work in the presence of an inert organic solvent. These preferably include aliphatic, alicyclic or aromatic hydrocarbons, for example petroleum ether, hexane, heptane, cyclohexane, methylcyclohexane or decalin; halogenated hydrocarbons, for example chlorobenzene, dichlorobenzene, dichloromethane, chloroform, tetrachloromethane, dichloroethane or trichloroethane.

When performing the process according to the invention, the reaction temperatures can be varied within a relatively wide range. In general, temperatures of 20° C. to 100° C., preferably temperatures of 30° C. to 80° C., more preferably temperatures of 30° C. to 60° C., are employed.

When performing the process according to the invention, generally between 0.8 and 10 equivalents, preferably between 1 and 5 equivalents, of exchangeable chlorine atoms in the form of a chlorinating agent are used per equivalent of 5-fluoro-1,3-dialkyl-1H-pyrazole-4-carbonyl fluoride of the formula (II). For instance, in the case of SiCl₄, 0.25 to 0.3 equivalent of this compound is needed for 1 equivalent of acid fluoride, and in the case of AlCl₃ correspondingly 0.3 to 0.4 equivalent.

The process according to the invention is generally carried out under standard pressure. However, it is also possible to work under elevated or reduced pressure—generally between 0.1 bar and 10 bar.

The 5-fluoro-1,3-dialkyl-1H-pyrazole-4-carbonyl chlorides of the formula (I) preparable by the process according to the invention are valuable intermediates for the preparation of fungicides (cf., for example, WO 03/010149).

The inventive preparation of 5-fluoro-1,3-dialkyl-1H-pyrazole-4-carbonyl chlorides of the formula (I) is described in the examples which follow, which further illustrate the above description. However, the examples should not be interpreted in a restrictive manner.

### PREPARATION EXAMPLES

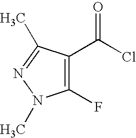

### Example 1

5-Fluoro-1,3-dimethyl-1H-pyrazole-4-carbonyl fluoride (7.21 g, 0.045 mol) and aluminium trichloride (60 mg, 0.45 mmol) are initially charged under argon. At 50° C., 2 g (0.012 mol) of silicon tetrachloride are added within 15 min, and the mixture is stirred at room temperature for 2 hours. In order to achieve complete conversion, another 1 g (0.006 mol) of silicon tetrachloride is added within 15 min, and the mixture is stirred at room temperature for a further 2 hours. The workup is effected by distillation. This affords 7.4 g (93% of theory) of 5-fluoro-1,3-dimethyl-1H-pyrazole-4-carbonyl chloride with a boiling point of 96-98° C. (at 9 mbar).

1. Process for preparing a 5-fluoro-1,3-dialkyl-1H-pyrazole-4-carbonyl chloride of formula (I) 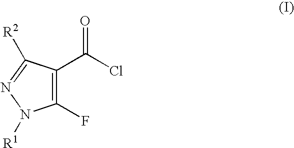
in which R¹ and R² are each independently C₁-C₃-alkyl, comprising: reacting
a 5-fluoro-1,3-dialkyl-1H-pyrazole-4-carbonyl fluoride of formula (II) 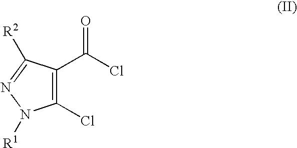
in
are reacted with a chlorinating agent, optionally in the presence of a diluent.

2. Process according to claim 1, wherein said fluoride of formula (II) comprises 5-fluoro-1,3-dimethyl-1H-pyrazole-4-carbonyl.

3. Process according to claim 1, wherein the chlorinating agent is at least one selected from the group consisting of silicon tetrachloride, trichloromethylsilane, dichlorodimethylsilane, trichlorophenylsilane, aluminium trichloride, boron trichloride, calcium chloride, titanium tetrachloride, tin tetrachloride, zinc dichloride and bismuth trichloride.

4. Process according to claim 1, wherein said process is effected in the absence of a diluent.

5. A starting material comprising
a 5-fluoro-1,3-dialkyl-1H-pyrazole-4-carbonyl fluoride of formula (II) 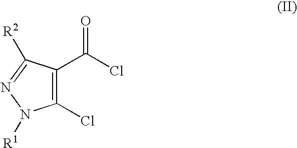
wherein R¹ and R² are each independently C₁-C₃-alkyl.

6. A fungicidal component comprising a chloride of formula (I) prepared according to a process as set forth in claim 1.

7. A process of claim 1 further comprising conducting said process at a temperature from 30-50° C.

8. A method for preparing a 5-fluoro-1,3-dialkyl-1H-pyrazole-4-carbonyl chloride of formula (I) 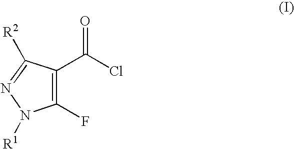
in which R¹ and R² are each independently C₁-C₃-alkyl, comprising a one step fluorine chlorine exchange.

9. A process of claim 2 further comprising conducting said process at a temperature from 30-50° C.

10. A process of claim 3 further comprising conducting said process at a temperature from 30-50° C.

11. A process of claim 4 further comprising conducting said process at a temperature from 30-50° C.
